(19) United States
(12) Patent Application Publication
(10) Pub. No.: US 2021/0092892 A1
(43) Pub. Date: Apr. 1, 2021

(54) Apparatus And Method For Soil Cultivation

(71) Applicant: Geoprospectors GmbH, Traiskirchen (AT)

(72) Inventor: Michael Pregesbauer, Baden (AT)

(21) Appl. No.: 16/966,135

(22) PCT Filed: Feb. 11, 2019
(86) PCT No.: PCT/EP2019/053233

§ 371 Date: Nov. 30, 2020

(30) Foreign Application Priority Data
Feb. 14, 2018 (AT) A 50135/2018

(51) Int. Cl. A01B 63/111; A01B 79/00; G01N 33/24

(52) U.S. Cl. CPC A01B 63/1112; G01N 2033/245; G01N 33/24; A01B 79/005

(57) ABSTRACT

A device for soil cultivation has a first sensor for detecting the condition of the soil and a controllable soil cultivation tool. The first sensor is designed in such a way that the condition of the soil can be determined prior to the cultivation by the soil cultivation tool. The present teaching further relates to an agricultural implement, including the device as well as a method for soil cultivation.

### TECHNICAL FIELD

The present teaching relates to a device for soil cultivation, comprising a first sensor for detecting the condition of the soil and a controllable soil cultivation tool, wherein the first sensor is designed in such a way that the condition of the soil can be determined prior to the cultivation by the soil cultivation tool.

### BACKGROUND

From the prior art, different devices and methods for variable soil cultivation are known. The cultivation depth of the soil cultivation tools in question, such as a cultivator, a subsoiler, a plough or other tools, are set on the basis of external information. This external information is preferably information that is provided by a sensor. This information serves for the determination of soil parameters which are relevant for the soil cultivation carried out. Sensors from the prior art have made it possible to detect this soil information in real time or to access information on the soil, which exist in the form of maps, for example.

Based on this information on the soil condition, the soil cultivation tool may be controlled and, thus, the desired cultivation depth, for example, or other cultivation parameters can be controlled by transmitting a manipulated variable to the soil cultivation tool, such as the control of a hydraulic cylinder for controlling the penetration depth of a ploughshare.

In the devices and methods for soil cultivation known from the prior art, the actually achieved work success is not determined so that the manipulated variable has to be readjusted constantly as a function of the soil conditions.

### SUMMARY

One object of the present teaching is to overcome the disadvantages of the prior art and to create a device for soil cultivation in which the manipulated variable of the soil cultivation tool is controlled as a function of the work success achieved.

The object is solved by the characterizing features of the present teaching.

The present teaching provides that a second sensor for the detection of the soil condition is provided, wherein the second sensor is designed in such a way that the soil condition can be determined after the cultivation by the soil cultivation tool and that a closed loop with a closed-loop control unit is formed, which is designed to determine a control variable for controlling the soil cultivation tool in real time as a function of the soil condition detected by the first sensor and the second sensor.

By determining the soil condition with the second sensor after the soil cultivation, the cultivation success caused by the soil cultivation tool may be determined.

The data of the first sensor and the second sensor is supplied to a closed-loop control unit, thus forming a closed loop. It is thus possible, to set a control variable, which is consequently used for the control of the soil cultivation tool and the determination of the manipulated variable.

Optionally, it may be provided that the first sensor is designed for the preferably contactless measurement or determination of soil parameters such as, for example, the electrical conductivity, radioactivity, compaction, texture and/or relative humidity of the soil, and is in particular an inductive sensor with a transmitting coil and a receiving coil, a magnetic sensor and/or a radiation detector.

The contactless measurement of the soil parameters guarantees that the first sensor is mechanically strained as little as possible. Different types of sensors can be used depending on the soil parameter to be measured.

Optionally, it may be provided that the second sensor is designed for the preferably contactless measurement or determination of a cultivation parameter, in particular the surface roughness of the soil, and is in particular a radar sensor, preferably a microwave radar sensor.

A radar sensor, preferably a microwave radar sensor, may optionally be used to determine the surface roughness of the soil. Radar-based methods are preferable to other distance measuring methods or imaging techniques since the radar measurement is largely unaffected by external influences such as dust generation.

Optionally, it may be provided that the closed-loop control unit is designed to determine a desired value of a cultivation parameter, for example a desired value of the surface roughness, from the soil parameters detected by the first sensor, preferably taking into account model parameters.

By using the data from the first sensor, the closed-loop control unit may optionally determine a desired value of a cultivation parameter. Preferably, this is done under consideration of model parameters, which can be read from a database, for example.

Optionally, it may be provided that the closed-loop control unit is designed to compare the desired value of the cultivation parameter to the actual value of the cultivation parameter detected by the second sensor and to determine therefrom the control variable for controlling the soil cultivation tool.

Optionally, it may also be provided that the closed-loop control unit can also process the data from the second sensor. By comparing the actual value to the desired value a control variable is determined as a function of the actual cultivation success.

Optionally, it may be provided that an implement control unit is provided, which is designed to determine a manipulated variable for controlling the soil cultivation tool from the control variable supplied by the closed-loop control unit.

This manipulated variable, for example the deflection of a hydraulic cylinder or an electric stepper motor, may differ from the actual condition, for example the actual penetration depth, of the soil cultivation tool due to the actual soil conditions and the actual environmental conditions. To this end, it may be provided that the actual penetration depth is determined by measuring the actual deflection of the actuators of the soil cultivation tool, such as the hydraulic cylinders or stepper motors, for example, in a measuring device on the soil cultivation tool. On the soil cultivation tool, a closed loop can be established between the desired penetration depth and the actual penetration depth of these actuators, wherein the difference between the two values is kept as small as possible and preferably results in the value zero. If the soil conditions or the environmental conditions change, the manipulated variable of the soil cultivation tool is adjusted accordingly.

The implement control unit serves the control of the soil cultivation tool. Optionally, single modules of the soil cultivation tool, such as different hydraulic cylinders, can be controlled with different manipulated variables.

Optionally, it may be provided that the soil cultivation tool is a soil cultivation tool which can be controlled hydraulically and has at least one hydraulic cylinder.

If the soil cultivation tool has at least one hydraulic cylinder, the actual penetration depth of the soil cultivation tool can be determined in particular by the deflection of the hydraulic cylinder. Optionally, this actual penetration depth of the soil cultivation tool may be returned to the closed loop and be used for the control.

Optionally, it may be provided that a measuring device is provided for measuring the lengths of the hydraulic cylinder.

Optionally, it may be provided that the soil cultivation tool is designed as a cultivator, subsoiler, plough or the like.

Optionally, it may be provided that multiple second sensors are provided, which are arranged in particular in the form of a sensor array, preferably in the form of a fan array. The sensor array may in particular be arranged in a direction transversal to the direction of travel.

If multiple second sensors are provided, an average value of the results obtained by the sensors may for example be used to determine the actual value of the cultivation parameter. This way, a location-dependent fluctuation of the measured values can be reduced. However, it may optionally also be provided that the data of the multiple second sensors is processed in a different way.

For example, it may be provided that different manipulated variables are assigned to different hydraulic cylinders as a function of data of different second sensors.

The present teaching also relates to an agricultural implement, comprising a device, wherein in the intended operation of the agricultural implement, the first sensor is arranged in the front region of the agricultural implement, with respect to the direction of travel, and the soil cultivation tool is arranged in the rear region or behind the agricultural implement, with respect to the direction of travel.

The present teaching also relates to a method for controlling the cultivation of a soil in real time, comprising the steps of:

detecting soil parameters of the soil with a first sensor prior to cultivating the soil with a soil cultivation tool,

determining a desired value of a soil cultivation parameter on the basis of the soil parameters obtained in step one, preferably taking into account model parameters,

detecting an actual value of a soil cultivation parameter with a second sensor after the cultivation of the soil with a soil cultivation tool,

feeding back the actual value of the cultivation parameter detected by the second sensor to a closed-loop control unit, comparing it with the desired value of the cultivation parameter and determining a control variable for controlling the soil cultivation tool,

determining a manipulated variable from the control variable in an implement control unit for controlling the soil cultivation tool, and

controlling the soil cultivation tool with the manipulated variable and, optionally, measuring the properties of the soil cultivation tool with a measuring device.

Optionally, it may be provided that the surface roughness of the soil is determined with the second sensor, and that a parameter value is determined as a function of the surface roughness.

Optionally, the surface roughness may be used as a measure of the quality of a decompaction process. For example, a lower surface roughness can be used as an indication of good cultivation success.

Optionally, it may be provided that a classification of the cultivation quality of the soil is carried out with the data of the second sensor.

Optionally, it may be provided that the method is carried out during a working travel of an agricultural implement, wherein the surface of the soil is scanned with the first sensor and the second sensor in the direction of travel of the agricultural implement.

Further features of the present teaching become apparent from the patent claims, the exemplary embodiments and the figures.

### BRIEF DESCRIPTION OF THE DRAWINGS

In the following, the present teaching is explained in more detail by means of a specific exemplary embodiment, wherein:

FIG. 1 shows a schematic view of an agricultural implement equipped with the device according to the present teaching;

FIG. 2 shows a schematic view of a closed loop used in the device according to the present teaching;

FIG. 3 shows a schematic view of a closed-loop control device used in the closed loop.

### DETAILED DESCRIPTION

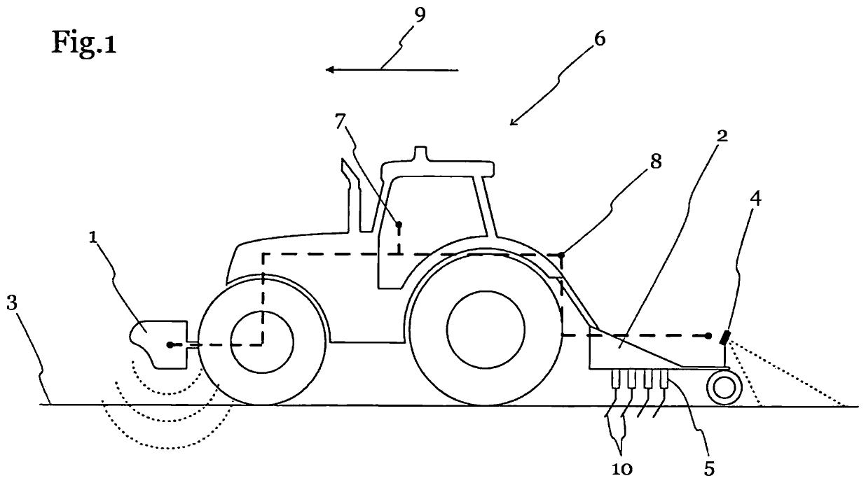

FIG. 1 shows a schematic view of an agricultural implement 6, on which a device according to the present teaching is arranged. The device comprises a first sensor 1, a closed-loop control unit 7, an implement control unit 8, a soil cultivation tool 2 equipped with hydraulic cylinders 5, and a second sensor 4. The soil cultivation tool is used for the cultivation of the soil 3.

The agricultural implement 6, which is represented as a tractor in this exemplary embodiment, moves over the soil 3 to an area to be cultivated in a direction of travel 9. The first sensor 1, in this exemplary embodiment an inductive sensor with an electromagnetic transmitting coil and an electromagnetic receiving coil, is used to determine the soil parameters in an uncultivated part of the soil 3. By means of the inductive sensor, the electrical conductivity of the soil can be determined, for example. In an exemplary embodiment which is not described, the first sensor 1 may also comprise a radioactivity sensor, for example. Multiple sensor types can also be combined. Soil parameters determined by the first sensor 1 may optionally comprise: density, humidity, surface roughness, without being limited to the soil parameters stated here.

The soil parameters determined by means of the first sensor 1 are transmitted to the closed-loop control unit 7 and a control variable is determined, optionally using stored model parameters. In this exemplary embodiment, this control variable is transmitted to the implement control unit 8 via an external cable connection. In another exemplary embodiment, the transmission can also take place via an already existing data bus of the agricultural implement 6.

The implement control unit 8 determines a manipulated variable from the control variable and forwards it as a desired value to the soil cultivation tool 2. Since this exemplary embodiment concerns a soil cultivation tool with hydraulic cylinders 5, the manipulated variable is substantially to be understood as the pressure with which the hydraulic cylinders 5 are pressurized.

In this exemplary embodiment, the same control variable is transmitted to all hydraulic cylinders 5. In other exemplary embodiments, however, it may be provided that the implement control unit 8 assigns each hydraulic cylinder 5 its own control variable, which optionally differs from the others.

According to the control variable transmitted by the implement control unit 8, the extensions 10 arranged on the hydraulic cylinders 5 of the soil cultivation tool 2 penetrate into the soil 3. Optionally, however, it may not be possible to deflect the hydraulic cylinders to the desired depth under the specified pressure, for example because the resistance of the soil 3 is too big. In this case, the desired penetration depth differs from the actual penetration depth. In this exemplary embodiment, the actual penetration depth is determined by means of a measuring device, e.g. a distance measuring system, arranged on the hydraulic cylinders 5. This distance measuring system determines the deflection of the hydraulic cylinders 5 and thus derives the penetration depth of the extensions 10. In an exemplary embodiment which is not described it may be provided that the actual penetration depth of the extensions 10 returns to the closed loop. It may also be provided that the actual deflection or penetration depth, respectively, of the extensions is controlled by a separate, second closed loop on the soil cultivation tool or the implement control unit, respectively, to ensure that the actual penetration depth corresponds to the value of the control variable.

When the soil 3 is cultivated with soil cultivation tool 2, a cultivated area of the soil 3 is left behind the soil cultivation tool 2 in the direction of travel 9 of the agricultural implement 6. The second sensor 4 is arranged on the soil cultivation tool 2 in such a way that it can analyze that area of the soil 3 which lies directly behind the soil cultivation tool 2. In this exemplary embodiment, the second sensor 4 is a microwave radar device. Since soil cultivation can cause a lot of dust, especially in the case of very dry soils, a radar-based sensor is used in this exemplary embodiment. In contrast to optical sensors, for example, this sensor is not affected by a possible dust generation.

In this exemplary embodiment the second sensor 4 is designed to measure the surface roughness. This is done in particular by determining the distance between the second sensor 4 and the surface of the soil 3 and by creating a topography profile from the data obtained. In the closed loop according to the present teaching, the actual value of the soil cultivation determined via the surface roughness, also referred to as the cultivation success, is transmitted to the closed-loop control unit 7. In the closed-loop control unit 7, a new control variable is determined by comparing the desired value with the actual value, which in turn is transmitted to the implement control unit 8.

In other exemplary embodiments which are not described, multiple second sensors 4 are used in the form of a fan array. In particular, in this exemplary embodiment, the second sensors 4 can be arranged in an orientation transversal to, in particular normal to, the direction of travel 9 and in a plane substantially parallel to the soil 3. This allows the reliability and accuracy of the determination of the surface roughness to be increased. Also, by using the data of multiple second sensors 4, multiple hydraulic cylinders 5 can be controlled independently.

The closed loop described above is continuously executed when the agricultural implement 6 moves, which allows the parameters to be adjusted in real time.

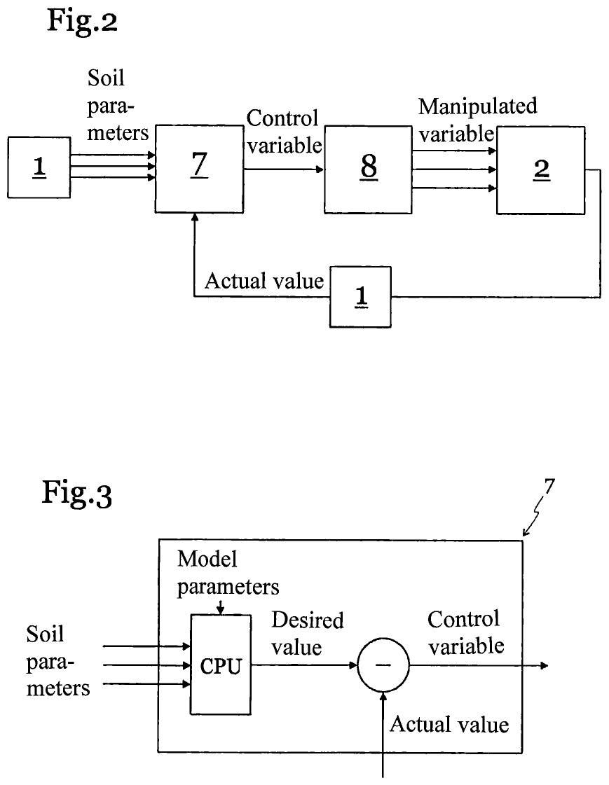

FIG. 2 shows a flow diagram of an exemplary embodiment of a closed loop used in the device according to the present teaching. As describe above, at least one soil parameter, for example the electrical conductivity or humidity, is determined by a first sensor 1 and transmitted to the closed-loop control unit 7. From the soil parameter, the closed-loop control unit 7 determines a control variable with the help of model parameters, which is forwarded to the implement control unit 8. Via the control variable transmitted by the closed-loop control unit 7 manipulated variables are set, which are forwarded to the soil cultivation tool 2.

The soil cultivation leads to a change in the roughness of the soil 3, which is monitored with the help of the second sensor 4. The actual value determined this way, in this exemplary embodiment the surface roughness of the soil 3, is transmitted to the closed-loop control unit and, together with the desired value, used for determining an updated control variable.

FIG. 3 shows a flow diagram of an exemplary embodiment of a closed-loop control unit 7 in detail. In a CPU, the soil parameters are converted into a desired value by using model parameters. The desired value is compared to the actual value and the information is forwarded to the implement control unit as a control variable. The controller itself may be designed as a proportional controller, integral controller, differential controller, or a combination of these types of controllers, for example.

1. A device for soil cultivation, comprising
a first sensor for detecting the condition of the soil, and
a controllable soil cultivation tool,
wherein the first sensor is designed in such a way that the condition of the soil can be determined prior to the cultivation by the soil cultivation tool,
wherein a second sensor is provided for detecting the condition of the soil, the second sensor being designed in such a way that the soil condition can be determined after the cultivation by the soil cultivation tool, and a closed loop with a closed-loop control unit is formed, which is designed to determine a control variable for controlling the soil cultivation tool in real time as a function of the soil condition detected by the first sensor and the second sensor.

2. The device according to claim 1, wherein the first sensor is designed for contactless measurement or determination of soil parameters.

3. The device according to claim 1, wherein the second sensor is designed for the contactless measurement or determination of a cultivation parameter.

4. The device according to claim 1, wherein the closed-loop control unit is designed to determine a desired value of a cultivation parameter.

5. The device according to claim 4, wherein the closed-loop control unit is designed to compare the desired value of the cultivation parameter to the actual value of the cultivation parameter detected by the second sensor and to determine therefrom the control variable for controlling the soil cultivation tool.

6. The device according to claim 1, wherein an implement control unit is provided, which is designed to determine a manipulated variable for controlling the soil cultivation tool from the control variable supplied by the closed-loop control unit.

7. The device according to claim 1, wherein the soil cultivation tool comprises a hydraulically controllable soil cultivation tool with at least one hydraulic cylinder.

8. The device according to claim 7, wherein a measuring device is provided for measuring the length of the hydraulic cylinder.

9. The device according to claim 1, wherein the soil cultivation tool is designed as a cultivator, subsoiler, or plough.

10. The device according to claim 1, wherein multiple second sensors are provided, which are arranged in particular in the form of a sensor array.

11. An agricultural implement, comprising a device according to claim 1, wherein in the intended operation of the agricultural implement, the first sensor is arranged in the front region of the agricultural implement, with respect to the direction of travel, and the soil cultivation tool is arranged in the rear region or behind the agricultural implement, with respect to the direction of travel.

12. A method for controlling the cultivation of a soi1 in real time, comprising:
detecting soil parameters of the soil with a first sensor prior to cultivating the soil with a soil cultivation tool,
determining a desired value of a cultivation parameter on the basis of the soil parameters,
detecting an actual value of a cultivation parameter of the soil with a second sensor after the cultivation of the soil with the soil cultivation tool,
feeding back the actual value of the cultivation parameter detected by the second sensor to a closed-loop control unit, comparing it with the desired value of the cultivation parameter and determining a control variable for controlling the soil cultivation tool,
determining a manipulated variable from the control variable in an implement control unit for controlling the soil cultivation tool,
controlling the soil cultivation tool with the manipulated variable.

13. The method according to claim 12, wherein the surface roughness of the soil is determined with the second sensor, and a parameter value is determined as a function of the surface roughness.

14. The method according to claim 12, wherein a classification of a cultivation quality of the soil is carried out with the data of the second sensor.

15. The method according to claim 12, wherein the method is carried out during a working travel of an agricultural implement, wherein a surface of the soil is scanned with the first sensor and the second sensor in the direction of travel of the agricultural implement.

16. The method according to claim 12, further comprising measuring properties of the soil cultivation tool with a measuring device.

17. The method according to claim 12, wherein the determining a desired value of a cultivation parameter takes into account model parameters.

18. The device according to claim 1, wherein the first sensor comprises an inductive sensor with a transmitting coil and a receiving coil.

19. The device according to claim 2, wherein the soil parameters comprise electrical conductivity, radioactivity, compaction, texture, and/or relative humidity of the soil.

20. The device according to claim 3, wherein the cultivation parameter comprises surface roughness of the soil.
